(19) United States
(12) Patent Application Publication
(10) Pub. No.: US 2020/0243169 A1
(43) Pub. Date: Jul. 30, 2020

(54) RESEARCH STUDY DATA ACQUISITION AND QUALITY CONTROL SYSTEMS AND METHODS

(71) Applicant: Invio, Inc., Seattle, WA (US)

(72) Inventors: J. Dmitri Poppa, Seattle, WA (US); Cassandra L. Wallender, Seattle, WA (US); Saqib Rokadia, Seattle, WA (US); Brian Caruso, Seattle, WA (US)

(21) Appl. No.: 16/726,133

(22) Filed: Dec. 23, 2019

Related U.S. Application Data
(63) Continuation of application No. 15/611,671, filed on Jun. 1, 2017, now abandoned.
(60) Provisional application No. 62/360,032, filed on Jul. 8, 2016.
(60) Provisional application No. 62/344,335, filed on Jun. 1, 2016.

(51) Int. Cl. G16H 10/20; G16H 10/60; G06F 16/23

(52) U.S. Cl. CPC G16H 10/20; G06F 16/2379; G16H 10/60

(57) ABSTRACT

Provided herein are systems and methods for clinical research study data acquisition and quality control.

### CROSS-REFERENCE TO RELATED APPLICATIONS

This application is a continuation of, and claims priority to, U.S. patent application Ser. No. 15/611,671, entitled “Research Study Data Acquisition and Quality Control Systems and Methods” and filed on Jun. 1, 2017 under Attorney Docket No. INVI-2016001f, the entire disclosure of which is hereby incorporated by reference for all purposes. This application is related to, and claims the benefit of, U.S. Provisional Patent Application 62/344,335, entitled “Client/Server-Based Research Study Data Acquisition and Quality Control Systems and Methods” and filed on Jun. 1, 2016 under Attorney Docket No. INVI-2016001Z, the entire disclosure of which, including the Appendices, is hereby incorporated by reference for all purposes. This application is related to, and claims the benefit of, U.S. Provisional Patent Application 62/360,032, entitled “Client/Server-Based Research Study Data Acquisition and Quality Control Systems and Methods” and filed on Jul. 8, 2016 under Attorney Docket No. INVI-2016001Z2, the entire disclosure of which, including the Appendices, is hereby incorporated by reference for all purposes.

### FIELD

The present disclosure relates to computing, and more particularly, to systems and methods for clinical study data acquisition and quality control.

### BACKGROUND

A clinical trial (also referred to as a “clinical study” and/or a “research study”) involves conducting investigative research using human subjects (also referred to as “participants” and/or “study participants”). A clinical trial may be conducted according to a predetermined research protocol, which may be set forth in a protocol document that describes the objective(s), design, methodology, statistical considerations, and organization of the trial. Clinical trials are designed to investigate questions about biomedical or behavioral interventions, such as treatments (e.g. novel vaccines, drugs, dietary choices, dietary supplements, and medical devices, or other products, treatment techniques or methodologies, and the like) and interventions. Clinical trials may generate data on safety and efficacy. In some circumstances, clinical trials may only be conducted after they have received health authority/ethics committee approval in the country where approval of the therapy is sought.

### Useful Definitions

Sponsor—A clinical trial sponsor may be an individual, company, institution, organization, or other entity that takes responsibility for the initiation, management, and/or financing of a clinical trial, e.g.: a pharmaceutical or medical device company or any company who has developed whatever medical innovation is being put through a clinical trial.

CRO/Contract Research Organization—A company that runs clinical trials on behalf of Sponsors; basically, outsourced clinical operations teams. A single CRO may have many sponsors as customers and may be running one or more trials for each sponsor.

CRA/Clinical Research Associate/Monitor—This person either works directly for the Sponsor or CRO. Their responsibility is to make sure Sites working for the Sponsor or CRO have submitted all appropriate documents and data. They also perform Site visits to do site selection and training, making sure each Site understands the Study Protocol.

Site/Research Site/Research Center—A hospital, clinic, or other location that has been contracted by the Sponsor or CRO to participate in the Study. It is their responsibility to enroll subjects at their Site into the study, follow the Study Protocol, and provide data and source documents to the Sponsor or CRO.

PI/Principal Investigator—The supervising medical doctor at a site. Their responsibilities may include seeing subjects, generating source documents, and review records for clinical significance.

Study Coordinator/Research Coordinator—A study coordinator may refer to one or more administrative staff members at a participating research site. A study coordinator may, for example, be responsible for taking source documents, extracting source data, and transcribing or otherwise loading the source data into CRFs. The person(s) at the site who runs the study there, they are often very busy enrolling subjects into the study and collecting study documents, making sure the PI has signed appropriate records, etc. Sometimes they are also involved in seeing subjects and directly generate source documents. They work with the monitor to make sure all the monitor's queries about the study documents are resolved.

Subjects—Participants in the study. Given clinical trials are medical research and not treatment, people enrolled in clinical trials at the sites are referred to as “Subjects” and not “Patients”.

IRB/Institutional Review Board—Each site either has a centralized or local ethics committee that reviews the Study Protocol and approves it before it can be put into practice at the Site(s) they represent. Their goal is to protect Subjects.

Study Protocol—The plan created by the Sponsor or CRO that outlines the study design, such as who should participate in the trial (inclusion and exclusion criteria) and the objective(s), design, methodology, statistical considerations, and organization of the trial.

Source Document(s)—Source documents may refer to original documents, data and records (including copies or transcriptions certified after verification as being accurate and complete, such as microfiches, photographic negatives, microfilm or magnetic media). Source documents may include hospital records; clinical and office charts; laboratory notes; memoranda; subjects' diaries of evaluation; checklists; pharmacy dispensing records; recorded data from automated instruments; x-ray films; participant/patient files; records kept at pharmacies, laboratories, and medico-technical departments involved in the clinical trial; papers, electronic records, and the like. They are the origin of data about the study.

Source Data—All information in original records and certified copies of original records of clinical findings, observations, or other activities (in a clinical investigation) used for the reconstruction and evaluation of the trial. Source data are contained in source documents (original records or certified copies).

Certified Copy—A copy (paper or electronic) of original information that has been verified, as indicated by a dated signature, as an exact copy, having all the same attributes and information as the original. Certified Copies are considered equivalent to Source Documents for the purpose of Source Data Verification.

Subject Binder—A subject binder may refer to a collection of source documents and other source data for an individual participant in a clinical trial (EMR & non-EMR source documents, such as questionnaires, external lab results, or research-related evaluations that may not be part of standard of care). A participant's subject binder may also contain other documents, such as checklists or worksheets, or participant informed consent forms or HIPAA authorizations for that participant. A site's set of records and Source Documents pertaining to a specific Subject for a specific Study. This is what monitors will access when they come on-site to do SDV. Today, often a physical 3-ring binder (or multiple binders).

Regulatory Binders—A site's set of records and the Study Protocol (and any revisions to that protocol) pertaining to a specific Study. Today, often a physical 3-ring binder (or multiple binders).

EHR/Electronic Health Record/EMR/Electronic Medical Record—An EHR system may refer to an electronic platform that contains individual electronic health records for patients and may be maintained by healthcare organizations and institutions. For example, a typical EHR system may include data corresponding to patients' medical history, diagnoses, treatment plans, immunization dates, allergies, radiology images, pharmacy records, and laboratory and test results. EHRs can be used by healthcare institutions to integrate real-time electronic healthcare information from medical devices and different health care providers involved in the care of patients. A site-specific system for patient records, owned by the Site. Sometimes, this data is pertinent to the study and included as Source Documents, however not all EHR data is applicable to the study and EHR access is tightly controlled. Also, as each site has their own EHR configuration, the data generated from one site's EHR is often inconsistent in structure with the data generated from another site's EHR. As a result of these issues, this data is today often printed and filed into a Subject Binder.

EDC/Electronic Data Capture—A study-specific system to house all Study data, owned and/or controlled by the study Sponsor or CRO. Study Coordinators transcribe data from Source Documents into the EDC, then Monitors check the EDC data against the Source Document Data in the process of SDV to ensure the accuracy of that transcription.

Optical character recognition/OCR—may refer to technology used to identify and/or convert images of typed, handwritten, printed text, and the like to machine-readable text.

Case Report Form/CRF—a printed, optical, or electronic document (sometimes referred to as an “eCRF” when electronic) designed to record protocol-related study data (also referred to as “clinical data”) to be reported to the sponsor on each trial subject. Simple versions of a CRF may consist of a spreadsheet or database. Study data in a CRF may be analyzed by the sponsor, sponsor representatives, and/or a regulatory agency (e.g. the United States Food and Drug Administration) to determine the efficacy and safety of the subject being evaluated during the clinical trial (such as a drug, device, or other investigational product or intervention being evaluated. Study data entered in a case report form may be transcribed, or interpreted, from source documents and source data gathered from evaluations or other interactions with study participants.

SDV/Source Data Verification—the monitoring process that a CRA performs to check that fields in the EDC match the original Source Documents stored in the Subject Binder.

Query—A query may refer to marking (or flagging) a data element, e.g. in a CRF, for notification and/or action, for example because the data element's value seems statistically unlikely (such as being outside of a predetermined reference range), is inconsistent with a corresponding source element value, does not make sense in the context of other data values (for example, a participant's cholesterol total having a value is less than the sum of the participant's LDL level and HDL level values). If a transcription error is found by a Monitor doing SDV, they query the originating site to correct. Monitors are never allowed to make corrections, only query the site for resolution.

### Clinical Trial Overview

Clinical trials can vary in size and cost, and can involve multiple research centers spread across wide geographic areas working independently. A clinical trial may be sponsored by a governmental organization or a pharmaceutical, biotechnology or medical device company. Certain functions necessary to the clinical trial, such as monitoring and lab work, may be managed by a third party, such as a CRO or a central laboratory.

Clinical trials of new drugs, for example, may be administered by a CRO hired by the trial sponsor. The trial sponsor may provide the drug being tested as well as medical oversight, while the CRO is contracted to perform all the administrative work during a clinical trial. The CRO may recruit, train, and supply participating sites, coordinate study administration and data collection, monitor participating research centers for compliance with the clinical protocol, audit research data, and ensure the trial sponsor receives research data collected from the research center.

At a participating research site, one or more research personnel, e.g. a research coordinator (also referred to as a study coordinator), may do most of the work in conducting the clinical trial. The research coordinator's job may include some or all of the following: working with the research site's IRB to obtain initial and ongoing permission to conduct the trial; assisting with administrative matters relating to the trial, identifying eligible patients; attempting to obtain consent for the identified eligible patients to participate in the trial; administering study treatment(s) to participants in the trial; obtaining source documents and/or source data relating to the trial; transcribing data; filing and/or archiving patient source documents and data; maintaining and updating study records, the sponsor, and/or the CRO.

A single clinical trial may cost hundreds of millions of dollars and a significant portion (often over 20%) of the cost may be incurred by monitoring the trial. As used herein, “monitoring” refers to the process of overseeing the progress of a clinical trial to ensure that it is conducted, recorded, and reported in accordance with the research protocol, standard operating procedures (SOPs), good clinical practice (GCP) standards, any applicable regulatory requirement(s), and the like. Monitoring tasks may be performed by a study monitor (also referred to as a clinical monitor, a clinical research monitor, a clinical research associate, a study site monitor, and/or a quality specialist). A significant portion of effort and cost of monitoring a clinical trial may be spent having study monitors traveling to and from research sites in order to manually verify trial data obtained by an onsite study coordinator via the use of the aforementioned physical 3-ring subject binders.

A conventional manual workflow for clinical trial data collection and monitoring may include:
(1) Obtaining participant informed consent and regulatory (e.g. HIPAA) authorization
(2) Evaluating participant for study visits and generating source documents as per the protocol or other trial requirements
(3) Enter relevant information on a Case Report Form (CRF), either on paper or in an EDC system. This may require:
   a. Identifying source documents containing source data or other information to extract from EMR and other sources.
   b. Manually transcribing data from source into corresponding study data fields on the CRF, creating study data
   c. Collating all source documents (and possible other subject information) into a subject binder
(4) CRF data may then be reviewed against source documents and data, per monitoring requirements:
   a. On-Site Monitoring may require:
      i. Monitor traveling to site.
      ii. Monitor working with Research Coordinator who provides access to Source Documents (e.g., via EMR, subject binder).
      iii. Monitor comparing source data with study data.
      iv. Monitor approving matching fields and flagging non-matching fields for query, which may be through the EDC, adding annotation in the EDC to explain the issues with non-matching fields, but may be done through other methods such as verbal communication, email notification, electronic messaging, or using paper forms.
      v. Monitor communicating need to contextualize or correct queried fields and/or values to the Research Coordinator.
      vi. Research Coordinator verifying issues and submitting additional context or change(s) if applicable with reason for change(s).
   b. Remote Monitoring may require:
      i. Monitor working with Research Coordinator or other site personnel who provides access to source documents (e.g., by login to cloud storage application, email, or fax).
      ii. Monitor comparing source data with study data manually field by field.
      iii. Monitor approving matching fields and flagging non-matching fields for query in the EDC, adding annotation in the EDC to explain the issues with non-matching fields.
      iv. Monitor communicating need to contextualize or correct queried fields to the Research Coordinator.
      v. Research Coordinator verifying issue and then submitting additional context or change(s), if applicable, with reason for change(s).
(5) Repeat steps 1 through 4 for some or all study data at some or all sites.

### BRIEF DESCRIPTION OF THE DRAWINGS

FIG. 1 illustrates exemplary network topologies of client/server-based research study data acquisition and quality control system in accordance with at least one embodiments.

FIG. 2 illustrates several components of an exemplary research coordinator client device in accordance with at least one embodiment.

FIG. 3 illustrates several components of an exemplary study monitor client device in accordance with at least one embodiment.

FIG. 4 illustrates several components of an exemplary server in accordance with at least one embodiment.

FIG. 5 illustrates a first exemplary series of communications between various components of a client/server-based research study data acquisition and quality control system in accordance with at least one embodiment.

FIG. 6 illustrates a second exemplary series of communications between various components of a client/server-based research study data acquisition and quality control system in accordance with at least one embodiment.

FIG. 7 illustrates an exemplary automatic case record validation flow diagram in accordance with at least one embodiment.

FIG. 8 illustrates an exemplary case record assisted review flow diagram in accordance with at least one embodiment.

FIG. 9 illustrates an exemplary case record field and/or case record value validation flow diagram in accordance with at least one embodiment.

FIG. 10 illustrates an exemplary mismatch review user interface in accordance with at least one embodiment.

FIG. 11 illustrates an exemplary out-of-bounds review user interface in accordance with at least one embodiment.

FIG. 12 illustrates an exemplary significant data review user interface in accordance with at least one embodiment.

FIG. 13 illustrates several components of an exemplary server in accordance with at least one embodiment.

### DESCRIPTION

The detailed description that follows is represented largely in terms of processes and symbolic representations of operations by conventional computer components, including a processor, memory storage devices for the processor, connected display devices and input devices. Furthermore, these processes and operations may utilize conventional computer components in a heterogeneous distributed computing environment, including remote file servers, computer servers, and/or memory storage devices. Each of these conventional distributed computing components is accessible by the processor via a communication network, which may include, but is not limited to, the Internet.

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise.

Various systems and methods embodying the present inventive concept are now described in detail with reference to the accompanying drawings. However, there is no intent to limit the scope of the inventive concept to the embodiments described herein. On the contrary, the intent is to provide a written description of embodiments that enable any person skilled in the art to make and use the described embodiments, as well as all alternatives, modifications, and equivalents encompassed by the inventive concept. The inventive concept underlying the present methods and systems relates to obtaining study data from source data or documents and the verification of transcribed study data against associated source data or documents, certain aspects of which are specifically defined by the Claims below. In various alternate embodiments, additional devices, may be added to, or combined with, the illustrated devices, and various illustrated devices may be combined into one or more other devices, without departing from the scope of the inventive concept.

### Example Network Topology of an Exemplary Clinical Trial Data Acquisition and Quality Control Systems and Methods

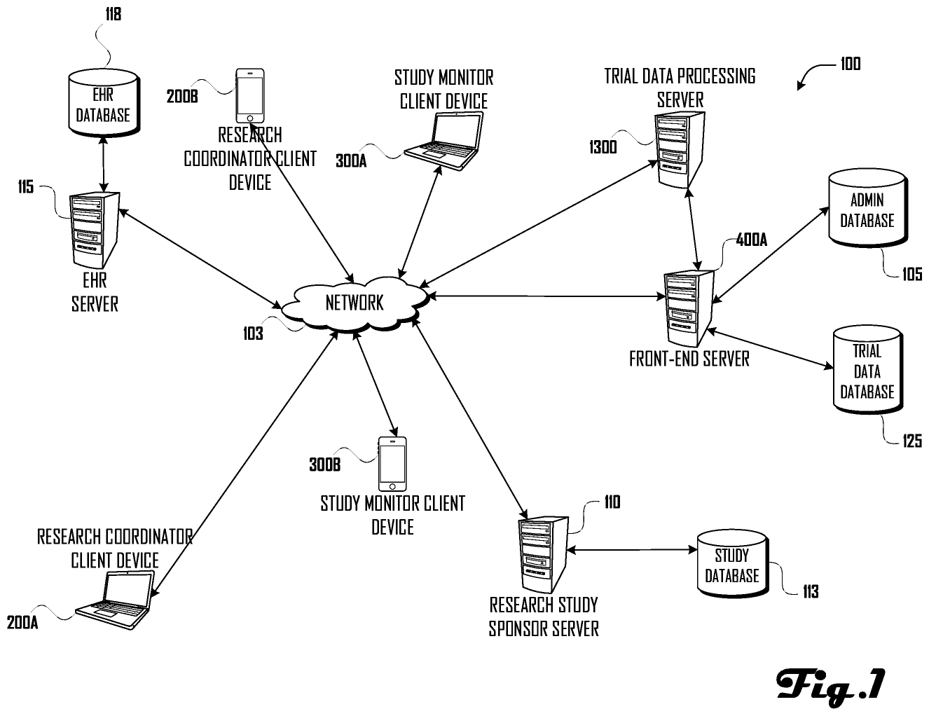

FIG. 1 illustrates an example network topology 100 of an exemplary clinical trial data acquisition and quality control system in accordance with various embodiments. Research coordinator client devices 200A-B, study monitor client devices 300A-B, and a front-end server 400A, may be in data communication with a network 103. In various embodiments, network 103 may include the Internet, one or more local area networks (“LANs”), one or more wide area networks (“WANs”), cellular data networks, and/or other data networks. Network 103 may, at various points, be a wired and/or wireless network. Front-end server 400A may be in data communication with a trial data processing server 1300 and an administrative database 105. In various embodiments, the administrative database 105 may be any of a variety of data storage mechanism, either contained in the font-end server 400A, as a stand-alone device (or devices), or on a remote device (or devices, such as a cloud storage system). Trial data processing server 1300 may be in data communication with a trial data database 125. A research study sponsor server 110 operated on behalf of a research study sponsor entity (not shown) and/or a contracted research organization (“CRO”) (not shown) may also be in data communication with network 103. A study database 113 may be in data communication with research study sponsor server 113. Research study sponsor server 110 may be an EDC.

The client/server-based research study data acquisition and quality control system illustrated in FIG. 1 may be operated in support of a research study system (not shown) operated on behalf of the research study sponsor entity and/or CRO. In some embodiments, the client/server-based research study data acquisition and quality control system may be a sub-system of such a research study system. In other embodiments, the client/server-based research study data acquisition and quality control system may operate independently of the research study system.

As is explained in more detail below, each of research study coordinator client devices 200A-B may be in the possession of and operated by a research coordinator (not shown) who may be an employee of a research site or facility (not shown) participating in a study conducted by the research study sponsor entity and/or CRO. Similarly, each of study monitor client devices 400A-B may in the possession of, and operated by, a clinical research assistant (“CRA”) (not shown) who may be an employee of the research study sponsor entity and/or CRO.

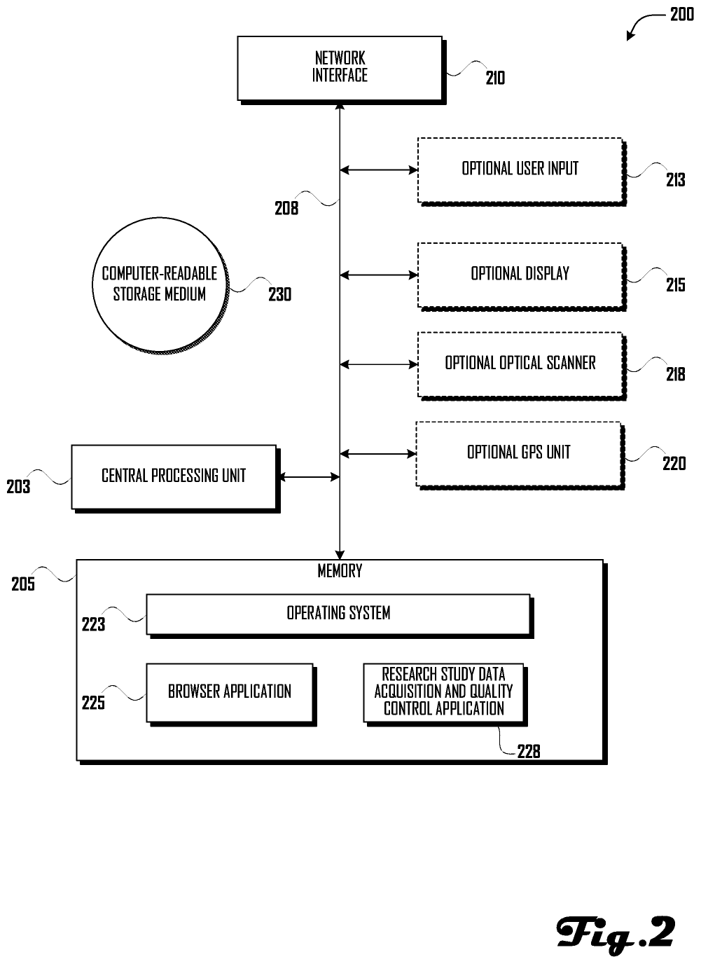

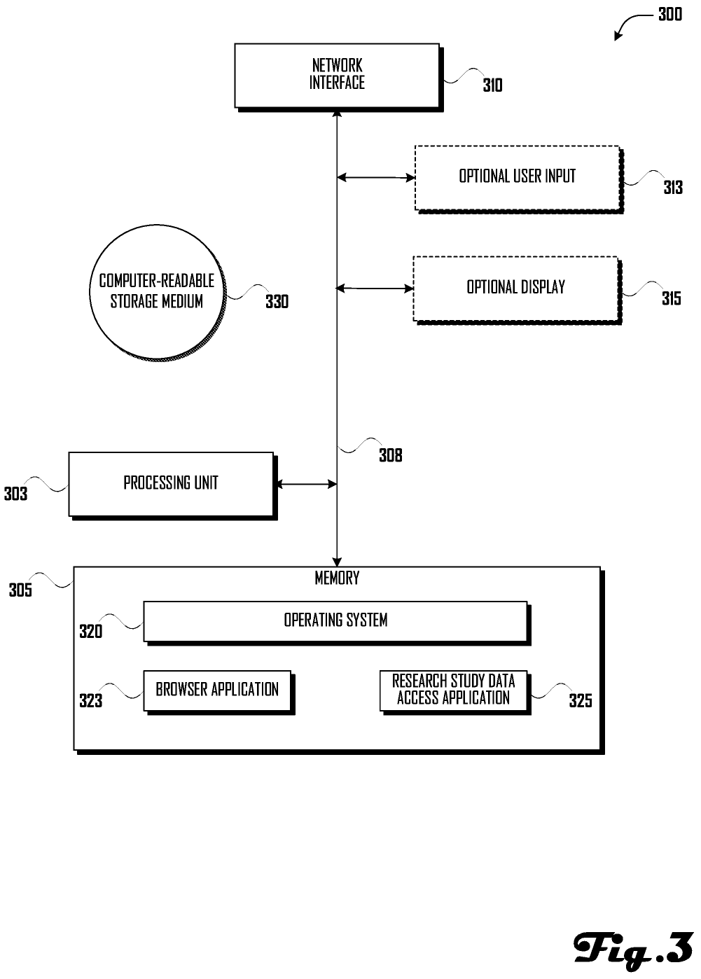

In these and other embodiments, a research coordinator client device 200, such as research coordinator client devices 200A-B, and/or a study monitor client device 300, such as study monitor client devices 300A-B, may be computing devices having form factors including general-purpose computers (including “desktop,” “laptop,” “notebook,” “tablet” computers, or the like); mobile phones; wearable computing devices (including watches, glasses, or the like); or the like. For simplified exemplary purposes, research study coordinator client devices 200A-B are depicted as having the form factor of laptop computers. The primary functional components of an exemplary, form-factor-independent research coordinator client device 200 are described below in reference to FIG. 2. For simplified exemplary purposes, study coordinator client devices 300A-B are depicted as having the form factors of a laptop computer and a mobile phone, respectively. The primary functional components of an exemplary, form-factor-independent study coordinator client device are described below in reference to FIG. 3.

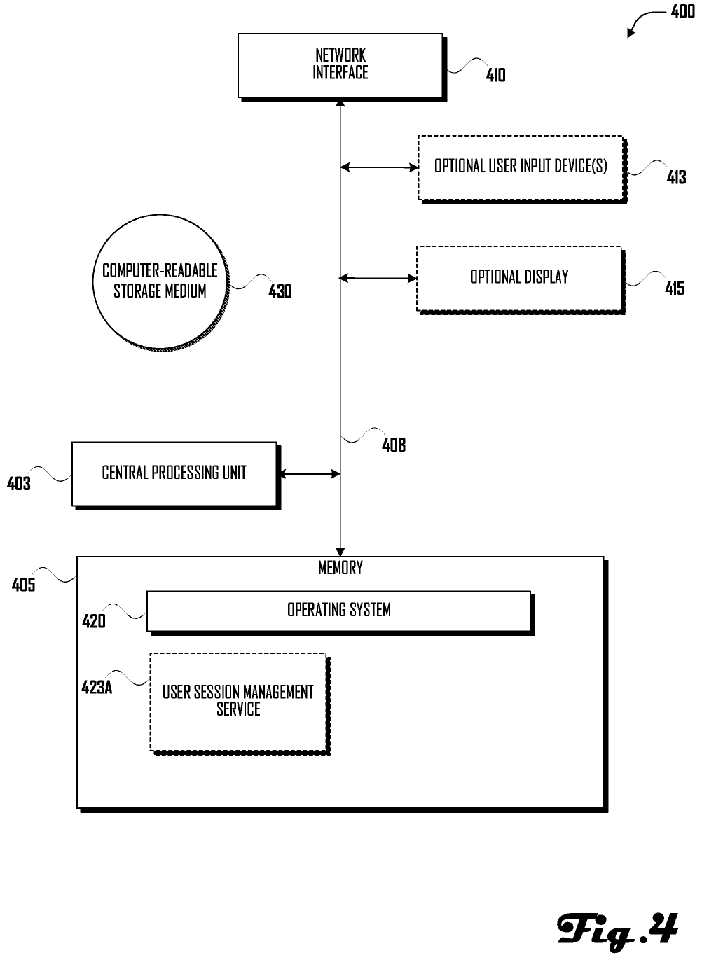

In various embodiments, front-end server 400A and trial data processing server 1300 (as well as research study sponsor server 110) may be networked computing devices generally capable of accepting requests over network 103, e.g. from research coordinator client device 200, each other, various databases, and/or other networked computing devices (not shown), and providing responses accordingly. The primary functional components of an exemplary server 400, such as front-end server 400A, are described below in reference to FIG. 4. In some embodiments, the Front-End Server 400A and Trial Data Processing Server 1300 may be implemented on a single device. While in other embodiments, Front-End Server 400A and Trial Data Processing Server 1300 may be implemented on multiple separate devices.

For the purposes of the present example, two research coordinator client devices 200 and two study monitor client devices 300 are shown, i.e. research coordinator client devices 200A-B and study monitor client devices 300A-B. In other embodiments, there may be many more research coordinator devices 200 and/or study monitor client devices 300.

Additionally, a research site EHR server 115, study monitor client devices 300A-B, front-end server 400A, and trial data processing server 1300 may be in data communication with network 103. An EHR database 118 may be in data communication with research site EHR server 115.

EHR server 115 and EHR database 118 may be operated in support of an EHR system (not shown). In various embodiments, the EHR system may be operated by a research site or may be operated independently of the research site. In various embodiments, EHR database 118 may contain source data for a clinical trial. EHR server 115 may provide the source data to trial data processing server 1300.

In various embodiments, a EHR server 115 may be a networked computing device generally capable of accepting requests over network 103, e.g. from trial data processing server 1300 and/or other networked computing devices (not shown), and providing responses accordingly.

### Exemplary Research Coordinator Client Device

Referring to FIG. 2, several components of an exemplary research coordinator client device 200, such as any of research coordinator client device 200A-B, is illustrated. In some embodiments, a research coordinator client device 200 may include many more components than those shown in FIG. 2. However, it is not necessary that all of these generally conventional components be shown in order to disclose an illustrative embodiment.

As shown in FIG. 2, exemplary research coordinator client device 200 includes a central processing unit 203 in data communication with memory 205 via a bus 208. Central processing unit 203 is an electronic circuit designed to carry out instructions of a computer program, e.g. obtained from memory 205, by performing the basic arithmetic, logical, control and input/output (I/O) operations specified by the program's instructions. Memory 205 generally comprises some or all of random access memory (RAM), read-only memory (ROM), and/or a permanent mass storage device, such as a disk drive, flash memory, or the like. Bus 208 is a communication system that transfers data between components within research coordinator client device 200, and encompasses any related hardware components (wire, optical fiber, etc.) and software, including communication protocols.

Research coordinator client device 200 may also include a network interface 210 for connecting to a network such as network 103, one or more optional user input device(s) 213, e.g. an alphanumeric keyboard, keypad, a mouse or other pointing device, a touchscreen, and/or a microphone, (or a user input port for connecting an external user input device), an optional display 215 (or a display port for connecting an external display device), an optional optical scanner 218 (or an accessory port for connecting an external optical scanner), an optional global-positioning-system (“GPS”) unit 220 (or an accessory port for connecting an external GPS device), and the like, all interconnected along with the network interface 210 via bus 208.

Memory 205 of exemplary research coordinator client device 200 may store program code, executable by central processing unit 203, corresponding to an operating system 223, as well as program code corresponding to various software applications, such as a browser application 225, a research study data acquisition and quality control application 228, and other software applications (not shown). Operating system 223 and various software applications may be loaded into memory 205 via network interface 210 or via a computer-readable storage medium 230, such as a hard-disk drive, a solid-state drive, an optical disc, a removable memory card, and/or the like.

Browser application 225 is a software application for retrieving, presenting, and traversing information resources on a network, such as network 103. Although browser application 225 may be primarily intended to use the World Wide Web, it may also be used to access information resources provided by remote servers in private networks. An information resource may be a web page, an image, a video, a document, or other piece of content and may be identified by a Uniform Resource Identifier (URI/URL) on network 103. An information resource may also provide browser application 225 executable program code for web applications, i.e. a software application that runs in and is rendered by browser application 225.

In operation, operating system 223 manages the hardware and software resources of research coordinator client device 200 and provides common services and memory allocation for various software applications, such as research study data acquisition and quality control application 228. For hardware functions such as network communications via network interface 210, receiving data via input 213, outputting data via optional display 215, and allocation of memory 205 for various software applications, such as research study data acquisition and quality control application 228, operating system 223 acts as an intermediary between software executing on the client device and the device's hardware.

For example, operating system 223 may cause a representation of available software applications, such as browser application 225 and research study data acquisition and quality control application 228, to be presented to a user of research coordinator client device 200 via display 215. If research coordinator client device 200 obtains an indication from a user, e.g. via user input 213, a desire to use research study data acquisition and quality control application 228, operating system 223 may instantiate a research study data acquisition and quality control application process (not shown), i.e. cause central processing unit 203 to begin executing the executable instructions of the research coordinator application and allocate a portion of memory 205 for its use.

In the case of a web application, browser application 225 may act as an intermediary between a software service operating on a remote server and the operating system 223. For example, a software service equivalent of research study data acquisition and quality control application 228 may be executing on front-end server 400A.

Although an exemplary research coordinator client device 200 has been described with hardware components that generally conforms to conventional general-purpose computing devices, a research coordinator client device may be any of a substantial number of devices capable of communicating with network 103 and executing instructions for performing research study data acquisition and quality control application 228.

### Exemplary Study Monitor Client Device

Referring to FIG. 3, several components of an exemplary study monitor client device 300, such as any one of remote study monitor client devices 300A-B, is illustrated. In some embodiments, a study monitor client device 300 may include many more components than those shown in FIG. 3. However, it is not necessary that all of these generally conventional components be shown in order to disclose an illustrative embodiment. As shown in FIG. 3, exemplary study monitor client device 300 may include a central processing unit 303 in data communication with memory 305 via a bus 308.

Central processing unit 303 is an electronic circuit designed to carry out instructions of a computer program, e.g. obtained from memory 305, by performing the basic arithmetic, logical, control and input/output (I/O) operations specified by the program's instructions. Memory 305 generally comprises some or all of random access memory (RAM), read-only memory (ROM), and/or a permanent mass storage device, such as a disk drive, flash memory, or the like. Bus 308 is a communication system that transfers data between components within study monitor client device 300, and encompasses any related hardware components (wire, optical fiber, etc.) and software, including communication protocols.

Study monitor client device 300 may also include a network interface 310 for connecting to a network such as network 103, one or more optional user input device(s) 313, e.g. an alphanumeric keyboard, keypad, a mouse or other pointing device, a touchscreen, and/or a microphone (or a user input port for connecting an external user input device), an optional display 315 (or a display-port for connecting an external display device), and the like, all interconnected along with the network interface 310 via bus 308.

Memory 305 of study monitor client device 300 may store program code, executable by central processing unit 303, corresponding to an operating system 320, as well as program code corresponding to various software applications, such as a browser application 323, research study data access application 325, and the like. Operating system 320 and various software applications, such as research study data access application 325, may be loaded into memory 305 via network interface 310 or via a computer-readable storage medium 330, such as a hard-disk drive, a solid-state drive, an optical disc, a removable memory card, and/or the like.

In operation, operating system 320 manages the hardware and software resources of study monitor client device 300 and provides common services and memory allocation for various software applications, such as research study data access application 325. For hardware functions such as network communications via network interface 310, receiving data via input 313, outputting data via optional display 315, and allocation of memory 305 for various software applications, such as research study data access application 325, operating system 320 acts as an intermediary between software executing on the study monitor client device and the device's hardware. For example, operating system 320 may cause a representation of available software applications, such as browser application 323 and research study data access application 325, to be presented to a user of client device 300 via display 315. If study monitor client device 300 obtains an indication from a user, e.g. via user input 313, a desire to use research study data access application 325, operating system 320 may instantiate a research study data access application process (not shown), i.e. cause central processing unit 303 to begin executing the executable instructions of the browser application and allocate a portion of memory 305 for its use.

Browser application 323 may be similar to browser application 225, described above. In the case of a web application, browser application 323 may act as an intermediary between a software service operating on a remote server and the operating system 320. For example, a software service equivalent of research study data access 325 may be executing on front-end server 400A.

Although an exemplary remote event/venue client device 300 has been described that generally conforms to conventional general-purpose computing devices, a remote event/venue client device may be any of a substantial number of devices capable of communicating with network 103 and executing instructions for performing browser application 323.

### Exemplary Server

Referring now to FIG. 4, several components of an exemplary server 400, such as front-end server 400A and trial data processing server 1300 (as well as other servers such as research study sponsor server 110 and EHR server 115) in accordance with at least one exemplary embodiment are illustrated. In some embodiments, a server 400 may include many more components than those shown in FIG. 4. However, it is not necessary that all of these generally conventional components be shown in order to disclose an illustrative embodiment. As shown in FIG. 4, a server 400 includes a central processing unit 403 and memory 405 connected by a bus 408.

Central processing unit 403 is an electronic circuit designed to carry out instructions of a computer program, e.g. obtained from memory 405, by performing the basic arithmetic, logical, control and input/output (I/O) operations specified by the program's instructions. Memory 405 may generally include some or all of random access memory (RAM), read-only memory (ROM), and/or a permanent mass storage device, such as a disk drive, flash memory, or the like. Bus 408 is communication system that transfers data between components within exemplary server 400, and includes any related hardware components (wire, optical fiber, etc.) and software, including communication protocols.

Server 400 may also include a network interface 410 for connecting to a network such as network 103, one or more optional user input device(s) 413, e.g. an alphanumeric keyboard, keypad, a mouse or other pointing device, a touchscreen, and/or a microphone (or a user input port for connecting an external user input device), and/or an optional display 415 (or a display port for connecting an external display device), both interconnected along with the network interface 410 via bus 408.

Memory 405 may store an operating system 420 and program code for various software services 423. For example, front-end server 400A may include executable instructions for performing user session management service 423A (indicated by dotted lines) and trial data processing server 1300 may include executable instructions for performing trial data processing service 1323.

Program code for these and other such software services, such as a software services (not shown) equivalent to research study data acquisition and quality control application 228 or research study data access application 325, may be loaded into memory 405 from a non-transient computer-readable storage medium 430 using a drive mechanism (not shown) associated with the non-transient computer-readable storage medium, such as, but not limited to, a DVD/CD-ROM drive, memory card, or the like. Software components may also be loaded into memory 404 via the network interface 410. A server 400 may also communicate via bus 408 with a database (not shown), such as admin database 105 and/or trial data database 125, or other local or remote data store.

Although an exemplary server 400 has been described having hardware components that generally conform to a conventional general-purpose computing device, a server may be any of a substantial number of devices capable of communicating with network 103 and executing instructions for performing user session management service 423A and/or trial data processing service 1323.

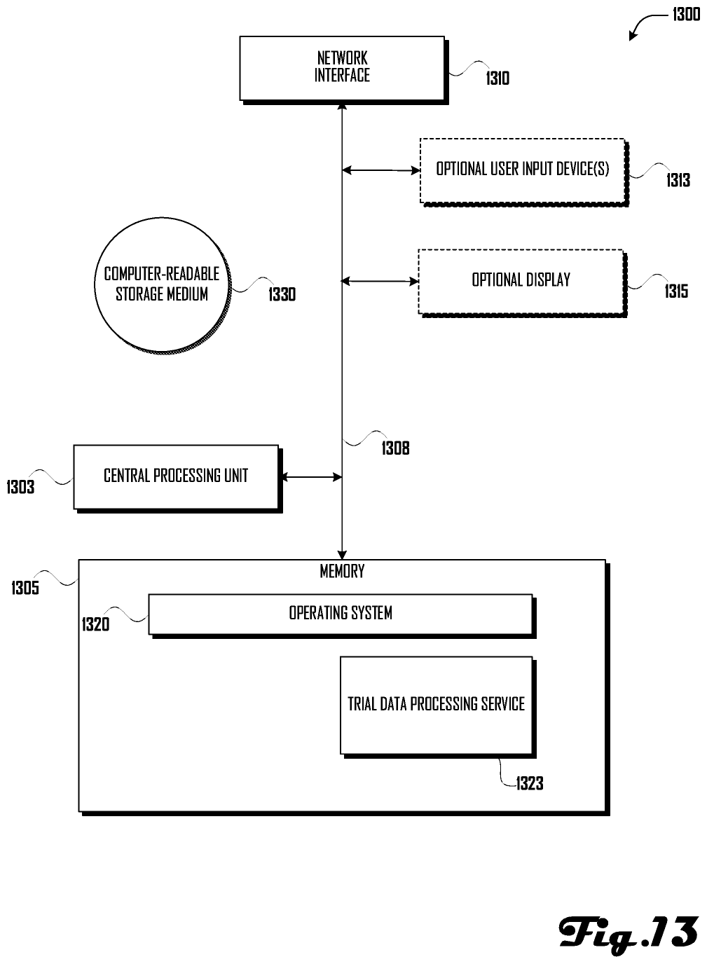

Referring now to FIG. 13, several components of a trial data processing server 1300 in accordance with at least one exemplary embodiment are illustrated. In some embodiments, a trial data processing server 1300 may include many more components than those shown in FIG. 13. However, it is not necessary that all of these generally conventional components be shown in order to disclose an illustrative embodiment. As shown in FIG. 13, a server 1300 includes a central processing unit 1303 and memory 1305 connected by a bus 1308.

Central processing unit 1303 is an electronic circuit designed to carry out instructions of a computer program, e.g. obtained from memory 1305, by performing the basic arithmetic, logical, control and input/output (I/O) operations specified by the program's instructions. Memory 1305 may generally include some or all of random access memory (RAM), read-only memory (ROM), and/or a permanent mass storage device, such as a disk drive, flash memory, or the like. Bus 1308 is communication system that transfers data between components within exemplary server 1300, and includes any related hardware components (wire, optical fiber, etc.) and software, including communication protocols.

Trial data processing server 1300 may also include a network interface 1310 for connecting to a network such as network 103, one or more optional user input device(s) 1313, e.g. an alphanumeric keyboard, keypad, a mouse or other pointing device, a touchscreen, and/or a microphone, (or a user input port for connecting an external user input device) and/or an optional display 1315 (or a display port for connecting an external display device), both interconnected along with the network interface 1310 via bus 1308.

Memory 1305 may store an operating system 1320 and program code for various software services and data storage. For example, trial data processing server 1300 may include executable instructions for performing trial data processing service 1323.

In one example embodiment, the trial data database 125 includes remotely accessible digital “binder” information for source data. The trial data database 125 would contain all of the source data for each participant in a clinical trial as well as relevant pieces of metadata (e.g.: tags, notes, and the like) that explain the participant's association with the trial. Some of the data that would be considered part of the subject binder can include, but are not limited to, the following:
Source data contained in EMR systems
Source data contained, but not externally accessible in EMR systems (e.g. printed from EMR via PDF)
Source data not contained in the EMR
Letters from healthcare professionals referring the participant to the trial
Signed and completed HIPAA waiver forms, allowing those performing the study to have access the participant's clinical data.

Trial data processing server 1300 would store the source data from the subject binder in a universally accessible location. As long as a user has credentials to access the trial data processing server 1300 and an internet connection, he or she can access the data in the subject binder from anywhere in the world.

In some embodiments, access to source data is restricted via business roles of users. In a physical setting, a research coordinator who had access to a physical subject binder for a particular participant would have access to all of the participant's data unless another person managing the trial manually sorted through the files first. In trial data processing server 1300, the research coordinators can be configured with a specific level of authorization, ensuring that the research coordinate can only view (and edit, when appropriate) source or clinical data which he or she has been granted access.

Program code for these and other such software services, such as a software services (not shown) equivalent to research study data acquisition and quality control application 228 or research study data access application 325, may be loaded into memory 1305 from a non-transient computer-readable storage medium 1330 using a drive mechanism (not shown) associated with the non-transient computer-readable storage medium, such as, but not limited to, a DVD/CD-ROM drive, memory card, or the like. Software components may also be loaded into memory 13013 via the network interface 1310. A server 1300 may also communicate via bus 1308 with a remote database (not shown), or other local or remote data store.

Although an exemplary server 400 has been described having hardware components that generally conform to a conventional general-purpose computing device, a server may be any of a substantial number of devices capable of communicating with network 103 and executing instructions for performing user session management service 423A and/or trial data processing service 1323.

In some embodiments, a server 400 may comprise one or more replicated and/or distributed physical or logical devices. In some embodiments, one or more of front-end server 400A and trial data processing server 1300 may be embodied by the same physical device.

### Research Study Data Acquisition via the Present Systems and Methods

Referring generally to FIGS. 1-4 and 13, above, in furtherance of the present methods and systems, a research coordinator assigned to collect data for a particular study (or studies) at a particular participating research site may interact with research study data acquisition and quality control application 228 (or a web application with similar functionality) operating on a research coordinator client device 200, such as research coordinator client device 200A-B.

In accordance with certain aspects of the present methods and systems, after identifying a source document, such as a medical record, patient intake form, questionnaire, or the like (in paper, digital, or other form), containing data relevant to a particular research study/clinical trial (“study data”), a research coordinator may transcribe the study data from the source document to a case report form specific to the research study.

In some embodiments, research study data acquisition and quality control application 228 may provide a user interface via display 215 having options for selectively (i) identifying a particular research study/clinical trial to associate with provided study data, (ii) identifying and accessing source data files containing study data stored locally on research coordinator client device 200, such as in memory 205 or in computer-readable storage medium 230, or stored remotely via network interface 210 and/or (iii) manually entering study data via user input 213. Research study data acquisition and quality control application 228 may further, e.g. upon selection of a particular research study/clinical trial and a source document, e.g. via user input 213, provide a digital copy of the selected source data file and a case report form associated with the selected research study/clinical trial, e.g. via display 215.

Upon selection of a source document, e.g. via user input 213, research study data acquisition and quality control application 228 may access the file, analyze the file, and attempt to identify and extract the relevant study data without additional input from the research coordinator. If research study data acquisition and quality control application 228 is unable to successfully identify and extract the relevant study data, additional input from the research coordinator may be obtained. For example, research study data acquisition and quality control application 228 may cause a representation of the file and an editable study data form to be displayed via display 215. The research coordinator may then manually copy the relevant study data from the representation of the file to the fillable study data form, e.g. via user input 213. If research study data acquisition and quality control application 228 is able to successfully identify and extract the relevant study data, the research study data acquisition and quality control application may still cause a representation of the file and an editable study data form to be displayed via display 215, thereby allowing the research coordinator to selectively validate the research study data identified and extracted by research study data acquisition and quality control application 228.

Research study data acquisition and quality control application 228 may interact with an EMR application (not shown), e.g. operating on 200 or another device in data communication with 200 via network 103, to obtain source documents. For example, research study data acquisition and quality control application 228 may present a list or directory of medical records stored in files accessible through the EMR application to the research coordinator via display 215 and the research coordinator may selectively indicate which medical records contain the desired study data, e.g. via user input 213. As above, research study data acquisition and quality control application 228 may then access the corresponding file, analyze the file, and attempt to identify and extract the relevant study data without additional input from the research coordinator. If research study data acquisition and quality control application 228 is unable to successfully identify and extract the relevant study data, additional input from the research coordinator may be obtained. For example, research study data acquisition and quality control application 228 may cause a representation of the file and an editable study data form to be displayed via display 215. The research coordinator may then manually copy the relevant study data from the representation of the file to the fillable study data form, e.g. via user input 213. If research study data acquisition and quality control application 228 is able to successfully identify and extract the relevant study data, the research study data acquisition and quality control application may still cause a representation of the file and an editable study data form to be displayed via display 215, thereby allowing the research coordinator to selectively check the study data identified and extracted by research study data acquisition and quality control application 228 for accuracy.

In accordance with certain embodiments, research study data acquisition and quality control application 228 may compare the source document to one or more predefined source document templates and provide mapping data indicating where in the source document the research study data acquisition and quality control application expects to find study data, e.g. via an overlay on the copy of the selected source data file being displayed or via providing a cropped image of that area. A research coordinator may then utilize the mapping coordinates to locate and identify the relevant study data from the copy of the source document and manually transcribe the source data into the case report form, or verify that the data already transcribed is correct or determine that the transcribed data is incorrect.

In accordance with other embodiments, research study data acquisition and quality control application 228 may compare the copy of the source document to predefined document templates to identify expected location of study data in the source document using predefined document templates, extract potential study data from the identified locations, and automatically transcribe the potential study data into the case report form. A research coordinator may then manually review the case report form to ensure the potential study data is actually study data and accurately reflects the data contained in the source document and edit the case report form, if necessary.

In accordance with other embodiments, research study data acquisition and quality control application 228 may analyze a plurality of source documents to identify potential study data without the use of predefined document templates, extract the potential study data, transcribe the potential study data to a case report form, analyze the potential study data for indications of invalidity (e.g. corrupted, fraudulent, or incorrectly identified data), and flag such indications, if any, for manual review by a research coordinator.

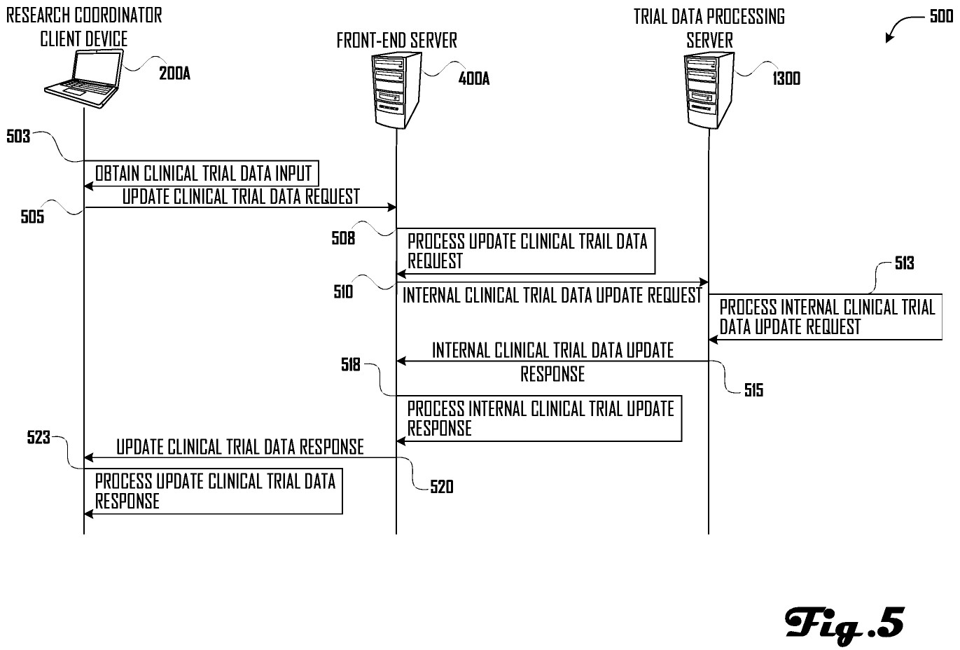

In conjunction with obtaining study data, such as in one or more of the manners described above, additional study metadata may be obtained and associated with the study data, such as an identifier associated with the research coordinator (an “RSC identifier”), an identifier associated the research site (a “site identifier”), the research study (a “study identifier”), the time and date the study data was initially recorded (“a data-recorded timestamp”), the time and date the data was obtained by research study data acquisition and quality control application 228 (a “data-entered timestamp”), an indication whether the underlying source of the study data (e.g. a case report form) was manually reviewed by a research coordinator (a “manual review flag”), an indication of how much time was spent manually reviewing the source of the study data (a “manual review value”), and/or the like. Research study data acquisition and quality control application 228 may then cause the study data, a copy of the underlying source of the study data (e.g. a digital copy of a case report form), and the associated metadata to be provided to trial data processing service 1323, for example via exemplary series of communications 500, described below in reference to FIG. 5.

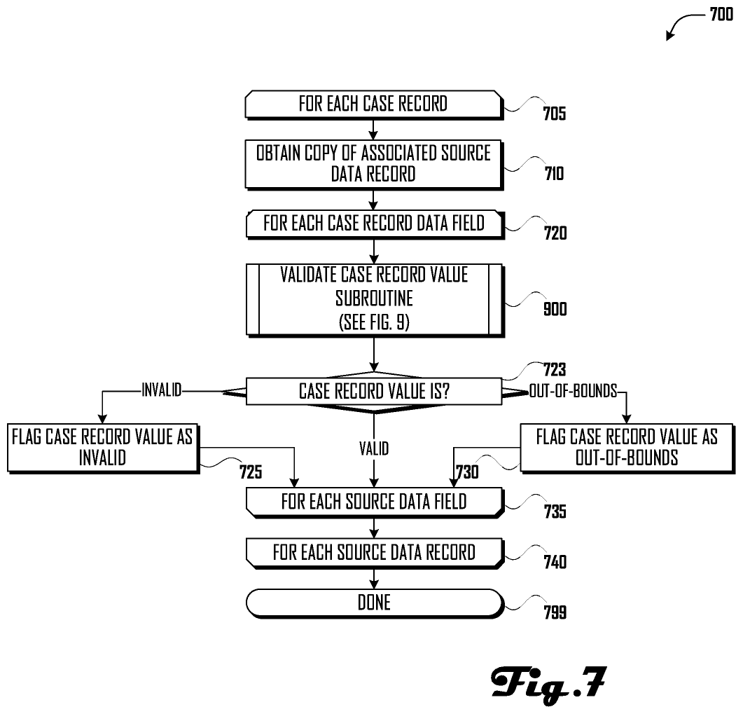

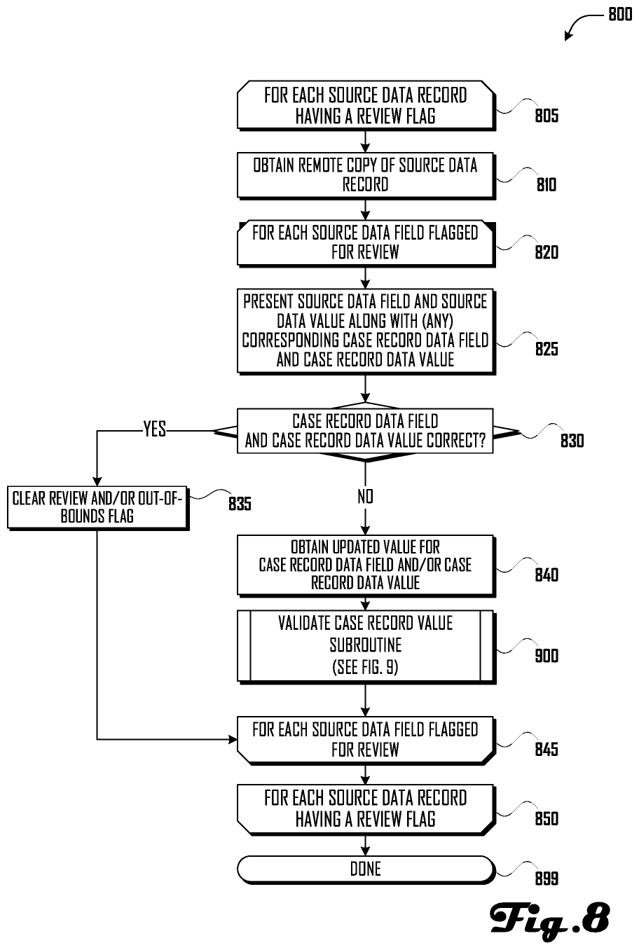

In accordance with other aspects of some embodiments, trial data processing service 1323 may, upon obtaining new study data and associated metadata, e.g. from an instantiation of research study data acquisition and quality control application 228, as described above: (i) determine which research study the data is associated with, e.g. via a study identifier provided with the study data; (ii) access research study records associated with the study identifier, e.g. stored in trial data database 125; (iii) validate the newly obtained research study data (e.g., via a case record validation routine 700 illustrated in FIG. 7); (iv) if errors are detected in the validation process, request manual review of some or all of the newly obtained research study data (e.g., via assisted validation routine 800 illustrated in FIG. 8); (v) combine the newly obtained and validated research study data with existing research study data, if any, in the research study records associated with the study identifier; and (vi) store the update research study records, e.g. in the trial data database.

Trial data processing service 1323 may provide study data updates to instantiations of research study data access application 325, e.g. operating on a study monitor client device 300, such as study monitor client device 300A-B. Such study data updates may be provided responsively upon request from research study data access application 325 and/or automatically at certain time intervals or when new study data is obtained by trial data processing service 1323. For example, the study data records associated with a particular study identifier may contain one or more associated study monitor identifiers, corresponding to study monitors assigned to the research study associated with the study identifier, and associated contact information (such as an email address). When a study monitor access research study data access application 325, research study data access application 325 may provide a notification to trial data processing service 1323, the notification including the monitor identifier associated with the study monitor. trial data processing service 1323 may then determine if an update should be provided, e.g. by looking up trial identifiers associated with the monitor identifier and searching for updated study data associated with the trial identifier that has been provided since the most recent update associated with the monitor identifier, and proceed accordingly. Such study data updates may include specific study data, associated metadata, cumulative summaries of study data, and/or links (e.g. URIs) to network locations where such specific study data and/or cumulative summaries may be accessed.

### Research Study Data Quality Control Via the Present Systems and Methods

Still referring generally to FIGS. 1-4 and in furtherance of the present methods and systems, some or all study data obtained by trial data processing service 1323 may need to be validated, e.g. in order to comply with various regulatory or sponsor requirements. In general, as used herein, the validation of study data refers to testing whether the study data obtained by trial data processing service 1323 matches the underlying source data (e.g. a comparison of source documents and/or data against study data, e.g. as may be contained in a case report form), and that the underlying source data is valid against any predetermined parameters that may exist. Various embodiments of the present methods and systems allow both automated and manual validation of study data obtained by trial data processing service 1323.

In certain embodiments, trial data processing service 1323 may analyze source documents, identify potential trial data within the source document, analyze the potential trial data, compare the potential trial data to the corresponding trial data contained in the associated case report form, and calculate a transcription confidence level representing an assessment of the accuracy of the trial data with respect to the underlying source data. If the transcription confidence level is below a pre-defined threshold value, e.g. less than 97%, or if the transcription is selected as part of a random sample, the trial data processing service 1323 may flag the trial data for manual validation, e.g. by a study monitor. If the transcription confidence level is above the pre-defined threshold value, or not selected as part of a sample, the trial data processing service 1323 may proceed with automated validation, the system will be allowed to validate the data on behalf of the end user.

For example, research study records associated with a particular trial identifier may include predetermined data ranges for study data. Upon obtaining new study data, trial data processing service 1323 may compare the obtained study data to the predetermined data ranges for the study data and, if the study data is outside the predetermined data range, the trial data processing service may flag the study data for further follow up/investigation.

Manual review of such flagged study data may reveal an error in the original data collection process (e.g. a transcription or OCR error between the original medical record and the case report form), an error in the data acquisition process (e.g. a transcription or OCR error between the case report form and the study data provided to trial data processing service 1323), or a data outlier (e.g. the study data is accurate but outside the predetermined range). In the latter case, the result of the manual review of the study data may override the automated validation result. In the former cases, a notification may be provided to the research coordinator responsible for the flagged study data so the flagged study data may be updated and re-validated.

In accordance with certain aspects of some embodiments, the study monitor may cause research study data access application 325 to obtain study data updates relating to research study data associated with one or more research studies/clinical trials. As noted above, such study data updates may include specific study data, associated metadata, cumulative summaries of study data, and/or links (e.g. URIs) to network locations where such specific study data and/or cumulative summaries may be accessed. For example, research study data access application 325 may provide a user interface via display 315 having options for selectively identifying and accessing research study records, e.g. stored in trial data database 125, associated with a particular study identifier. Upon selection of one or more research study records, either through manual selection by the study monitor via user input 313 or automatic selection by research study data access application 325 or trial data processing service 1323, research study data access application 325 and trial data processing service 1323 may cooperatively cause a selection of the study data associated with the trial identifier as well as a copy of the underlying source data to be presented via display 315. The study monitor may then selectively provide an indication of whether the study data accurately reflects the underlying source data. If the study monitor determines the study data does not accurately reflect the underlying source data, research study data access application 325 may provide options for flagging the discrepancy for investigation. Trial data processing service 1323 may then prepare a validation-fail notification and provide the validation-fail notification to the research coordinator associated with the study data.

### First Exemplary Series of Communications

FIG. 5 illustrates a first exemplary series of communications 500 between study monitor client device 200, front-end server 400A, and remote trial data processing server 1300 in accordance with various embodiments of an exemplary client/server-based research study data acquisition and quality control system, such as the exemplary client/server-based research study data acquisition and quality control system illustrated in FIG. 1.

Study monitor client device 200A may obtain 503 clinical trial data, e.g. via user input 213 in response to a prompt provided via display 215.

Study monitor client device 200A may provide front-end server 400A with a corresponding update clinical trial data request 505. Update clinical trial data request 505 may include user identifying information corresponding to a user of study monitor client device 200A (a monitor identifier), e.g. via an alphanumeric identifier associated with a site monitor; identifying information corresponding to a particular clinical trial (a trial identifier); a current location of study monitor client device 200A; clinical trial data, metadata; and the like.

Front-end server 400A may process 508 update clinical trial data request 505. For example, front-end server 400A may obtain additional metadata relating to the trial identifier from admin database 105 and/or other sources.

Front-end server 400A may provide trial data processing server 1300 with a corresponding internal clinical trial update request 510. Internal clinical trial data update request 510 may include data obtained from study monitor client device 200A via update clinical trial data request 505 and via processing 508 the update clinical trial data request by front-end server 400A.

Trial data processing server 1300 may process 513 internal clinical trial data update request 510. For example, trial data processing server 1300 may validate study data obtained from study monitor client device 200A, identify and flag questionable data, and update related clinical trial data records, e.g. in trial data database 125.

Trial data processing server 1300 may then provide an internal clinical trial data update response 515 to front-end server 400A.

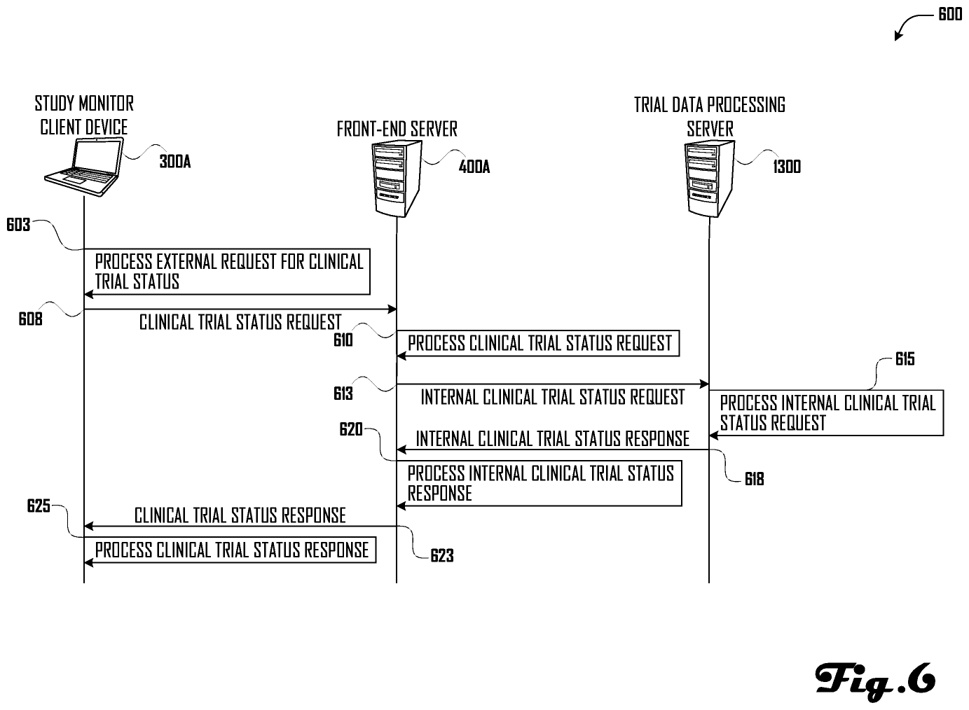

Front-end server 400A may then process 518 internal clinical trial data update response 515, e.g. by determining which research coordinator client device (see FIG. 6), if any, should be updated in response.

Front-end server 400A may then provide an update clinical trial data response 520 to study monitor client device 200A. Update clinical trial data response 520 may include queries corresponding to flagged data points, confirmation of the successful upload of the trial data contained in update clinical trial data request 505, error messages, and/or the like.

Study monitor client device 200A may then process 523 update clinical trial data response 520, for example by rendering information provided in the update clinical trial data response via display 215.

### Second Exemplary Series of Communications

FIG. 6 illustrates a second exemplary series of communications 600 between research coordinator client device 300A, front-end server 400A, and trial data processing server 1300 in accordance with various embodiments of an exemplary client/server-based research study data acquisition and quality control system, such as the exemplary client/server-based research study data acquisition and quality control system illustrated in FIG. 1.

Research coordinator client device 300A may process 603 an external request for a status update on a clinical trial, e.g. obtained via user input 313 in response to a prompt provided via display 315.

Research coordinator client device 300A may provide front-end server 400A with a corresponding clinical trial status request 608. Clinical trial status request 608 may include user identifying information corresponding to a user of research coordinator client device 300A (a coordinator identifier), e.g. via an alphanumeric identifier associated with a research coordinator; identifying information corresponding to a particular clinical trial (a trial identifier); and the like.

Front-end server 400A may process 610 clinical trial status request 608. For example, front-end server 400A may obtain metadata relating to clinical trial status request 608 from admin database.

Front-end server 400A may provide trial data processing server 1300 with a corresponding internal clinical trial status request 613. Internal clinical trial status request 613 may include data obtained from research coordinator client device 300A via clinical trial update request 608 and via processing 610 the clinical trial update request by front-end server 400A.

Trial data processing server 1300 may process 615 internal clinical trial status request 613 and provide trial status summary data, and/or the like.

Trial data processing server 1300 may then provide an internal clinical trial status response 618 to front-end server 400A, e.g. including the trial status summary data.

Front-end server 400A may process 620 internal clinical trial status response 618. For example, front-end server 400A reformat the trial status summary data based on preference data associated with the coordinator identifier provided in clinical trial status request 608.

Front-end server 400A may then provide a clinical trial status response 623 to research coordinator client device 300A, e.g. including the formatted trial status summary data.

Research study coordinator client device 300A may then process 625 clinical trial status response 623, for example by rendering the trial status summary data via display 215.

In order for the trial data processing server 1300 to assist monitors in reviewing case record files, the case record files are automatically compared to research study source data. In one embodiment, the source data (or possible certified copies of the source data) is converted into a machine-readable format (e.g., via voice recognition, optical character recognition, EHR ingestion, and the like).

FIG. 7 illustrates an exemplary automatic case record validation routine 700. Routine 700 begins at loop block 705 where for each case record a copy of an associated source data record is obtained in Block 710. In some embodiments, this may be a certified copy of the source data. Next in loop block 720 for each case record data field the case record validation subroutine 900 is called. If in decision block 703 it is determined that the case record data value is invalid then the case record data value is flagged as invalid at block 725. Alternately, if in decision block 723 it is determined that the case record value is out-of-bounds, then in Block 730 the case record data value is flagged as out-of-bounds. If the case record data value is determined to be valid in decision block 723 and processing continues to loop block 735 and routine 700 loops for each source data field. After which, processing loops for each source data record in loping block 740 until processing completes in termination block 799.

While automatic case record validation routine 700 is shown flagging case record values as valid/invalid or out-of-bounds, many more possible flags or metatags may be applied in alternate embodiments. For example, a monitor may request that certain value ranges for particular types of source data values be flagged as “of interest” or “clinically significant” to thereby speed the review of the case records while the monitor may be engaged with reviewing other materials, sites, and/or subjects.

When a monitor is reviewing case records for accuracy and information of interest, it behooves them to have the more relevant case record data values prioritized for review. And if inaccuracies are found, having the ability to query for updates to the case record data value(s) and/or source data values because monitors do not have permission to modify case record data values or source data values themselves.

FIG. 8 illustrates an exemplary case record assisted review routine 800. Case record assisted review routine 800 begins at loop block 805 where for each clinical trial source data record having a review flag the process loops. At block 810 a copy of the source data record is obtained. In some embodiments, this may be a certified copy. In loop block 820 the routine 800 loops for each subject data field flagged for review. Next, in block 825 the source data field and source data value along with any corresponding case record data field and case record data value are presented for review. If it is determined in decision block 830 that the case record data fields and case record data value are correct, then in block 835 the review and/or out-of-bounds flags on the case record data field and/or case record data value are cleared and processing proceeded to loop block 845. If, on the other hand, in decision block 830 it is determined that the case record data field and/or the case record data value are not correct, then in block 840 a query is made to obtain an updated case record data field and/or case record data value. The updated value for the case record data field and/or case record data value is then processed in the case record validation subroutine 900, after which processing continues to loop block 845 and after all loops are completed processing proceeds to the next loop block 850. Once all loops have been completed, processing terminates at termination block 899.

By allowing the monitor to quickly focus on the case record data values that have been automatically identified as mismatched and/or of interest, the monitor is able to quickly determine which queries are necessary.

In some embodiments, the monitor may be presented with and/or control a confidence level that they wish to apply to automatically identified mismatches and/or items of interest. For example, if an OCR scan of a source data returns a confidence value of the OCR operation, the monitor may choose to only see OCR value within a certain confidence value range.

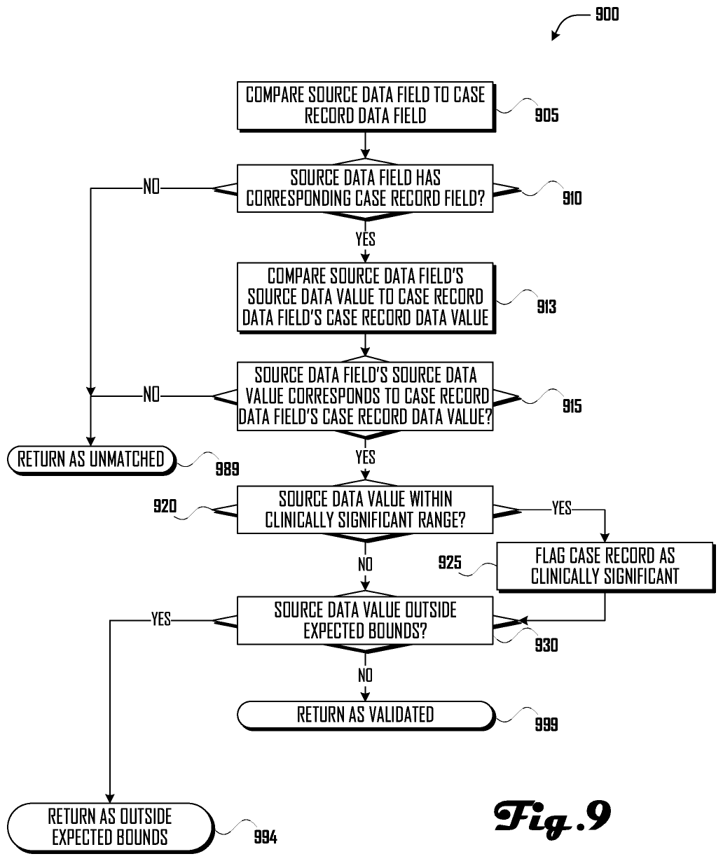

FIG. 9 illustrates an exemplary case record field and/or case record value validation subroutine 900 usable by various other routines. Beginning in block 905, a source data field is compared to an alleged associated case record data field. If it is determined in decision block 910 that the source data field corresponds to the case record field then processing continues to block 913. If on the other hand it is determined that the source data field does not have it corresponding case record field, then there is a good chance that the wrong source data and case record have been associated (possibly due to uploading the wrong document) and processing precedes to return block 989 where an unmatched value is returned.

In block 913 the source data field's source data value is compared to the case record data field' case record data value. Next, in decision block 915, if a determination is made that the source data value does not correspond to the case record data value, unmatched result is the returned in return block 989. On the other hand, if the source data value and the case record data value correspond, then a determination is made in decision block 920 if the source data value falls within a potentially clinically significant range. If the source value falls within the potentially clinically significant range, then in block 925 the case record (and possibly the case record data value) is tagged as potentially clinically significant and processing continues to block 930. If, in decision block 920 the source data value is determined not to be within a potentially clinically significant range processing continues to decision block 930 where determination is made whether the source data value is outside and predetermined bounds. If the source data value is outside of predetermined bounds then processing continues to return block 994 where a value of outside predetermined bounds is returned. Ultimately, if in decision block 930 it is determined that the source data value is not outside a predetermined range then processing proceeds to return block 999 where is a validated result is returned.

While an example case record field and/or case record value validation subroutine has been shown with example tests, other tests/validations/comparisons/reviews options may be present in other embodiments. For example, in some embodiments, there may be statistical testing for fraudulent or fabricated data.

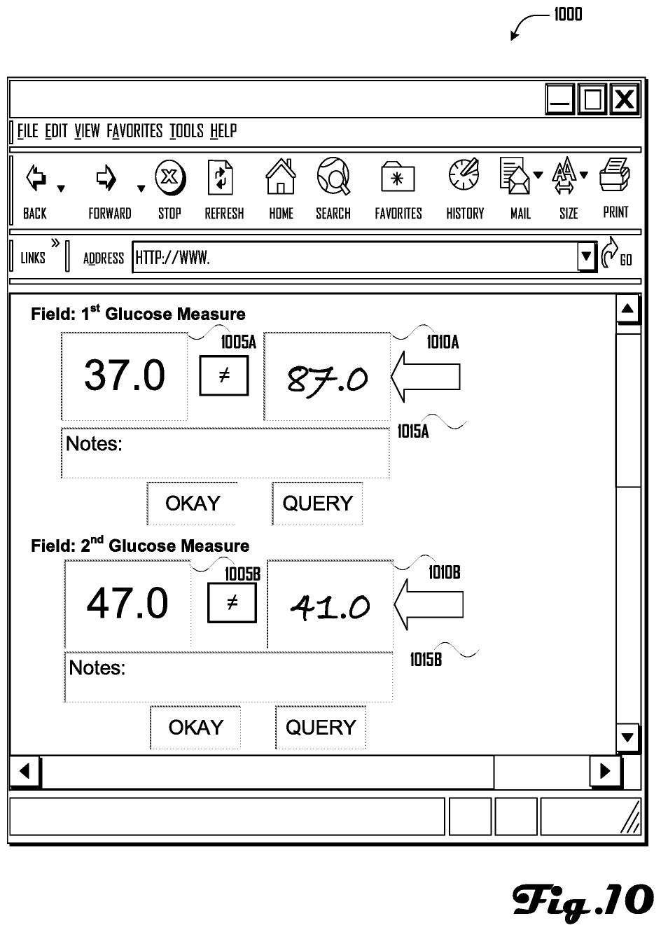

FIG. 10 illustrates an exemplary mismatch review user interface 1000. The mismatch review user interface 1000 includes a first case record value 1005A that is indicated as not being equal to an associated source data value 1010A. The source data value is highlighted from within a source view 1015A. The source user interface 1000 also includes a second case record value 1005B and a second source data value 1010B which are also indicated as being mismatched. The second source data value 1010B was highlighted from a second source view 1015B.

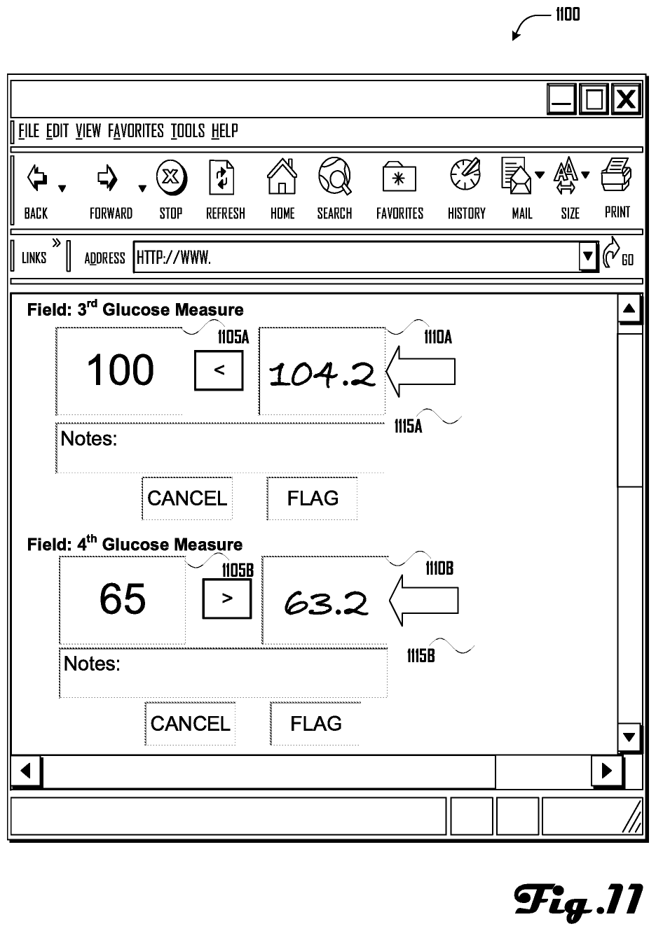

FIG. 11 illustrates an exemplary out-of-bounds review user interface 1100. The out-of-bounds review user interface 1100 includes a source data value 1110A that is indicated as being out-of-bounds with regards to a reference value 1105A. The source data value 1110A is highlighted from within a source view 1115A. The out-of-bounds review user interface 1100 also includes a second source data value 1110B that is indicated as being out-of-bounds with regards to a reference value 1105B. The source data value 1110B is highlighted from within a source view 1115B.

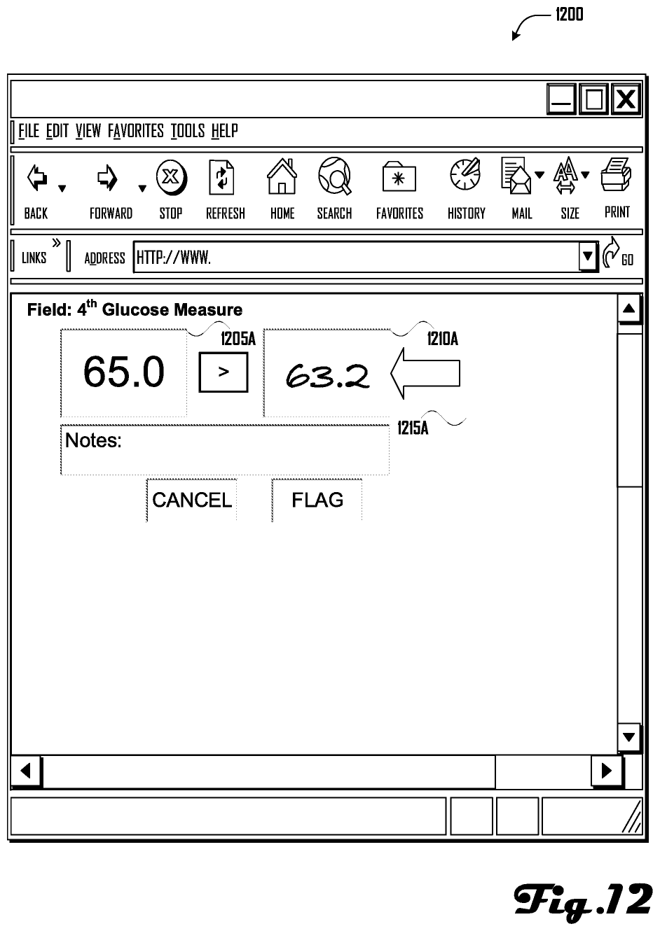

FIG. 12 illustrates an exemplary significant data review user interface 1200. the significant data review user interface 1200 includes a source data value 1210A that is beyond a significant reference value 1205A. The source data value 1210A is highlighted from within a source view 1215A.

Although specific embodiments have been illustrated and described herein, it will be appreciated by those of ordinary skill in the art that alternate and/or equivalent implementations may be substituted for the specific embodiments shown and described without departing from the scope of the present disclosure. For example, in some embodiments, instead of or in addition to the assisted data verification routine process described in verification routine 700 (see FIG. 7, discussed above), an automatic verification process may be used to automatically query based on mismatched source data and case record data. This application is intended to cover any adaptations or variations of the embodiments discussed herein.

1.-6. (canceled)

7. A clinical trial facilitated verification apparatus, comprising a processor and a memory comprising instructions for the processor to:
remotely obtain, by a study monitor client computing device remotely located from a research site, a digital case report form,
the digital case report form comprising a case record identifier, a first case record data field, and a first case record data value associated with the first case record data field;
remotely obtain, by the study monitor client computing device, a digital copy of a clinical trial source data record,
the clinical trial source data record comprising, in a non-machine-readable format, a subject identifier corresponding to said case record identifier, a first source data field allegedly corresponding to said first case record data field, and a first source data value allegedly corresponding to said first case record data value;
convert at least a portion of the digital copy of the clinical trial source data record into a machine-readable format, such that the first source data field and the first source data value are computer-recognized;
compare the computer-recognized version of said first source data field with said first case record data field;
determine that the computer-recognized version of said first source data field corresponds to said first case record data field;
compare the computer-recognized version of said first source data value with said first case record data value;
determine that the computer-recognized version of said first source data value does not correspond to said first case record data value;
present to a study monitor, via the study monitor client computing device, digital case report form data including at least one of said first source data value and said first case record data value; and
generate, on the study monitor client computing device, a remote visual mismatch indicator affiliated with at least one of said first source data field, said first source data value, said first case record data field, and said first case record data value,
such that the remote visual mismatch indicator distinguishes, among digital case report form record data values, a record data value that the computing system automatically identified as not corresponding to the clinical trial source data record.

8. The apparatus of claim 7, wherein the instructions to convert at least a portion of the digital copy of the clinical trial source data record into a machine-readable format are instructions to perform recognition selected from at least one of optical character recognition and voice recognition.

9. The apparatus of claim 7, wherein the computer-recognized version of the first source data field or value comprises transcription confidence data, and wherein said presenting digital case report form data to a study monitor further comprises, if the transcription confidence is below a pre-defined threshold value, presenting the transcription confidence data or flagging the digital case report form data for manual validation.

10. The apparatus of claim 7, where said digital copy of a clinical trial source data record comprises a certified digital copy of the clinical trial source data record.

11. The apparatus of claim 7, wherein the memory further comprises instructions for the processor to:
send, from the study monitor client computing device, an update request for said first case record data value;
obtain, by the study monitor client computing device, an updated digital case report form having an updated first case record data value;
compare the computer-recognized version of said first source data value with said updated first case record data value; and
determine that the computer-recognized version of said first source data value does correspond to said updated first case record data value.

12. The apparatus of claim 7, wherein the memory further comprises instructions for the processor to add a metatag to at least one of the digital case report form and said clinical trial source data record.

13. A non-transient computer-readable storage medium comprising clinical trial facilitated verification instructions, which, when executed by a processor:
remotely obtain, by a study monitor client computing device remotely located from a research site, a digital case report form,
the digital case report form comprising a case record identifier, a first case record data field, and a first case record data value associated with the first case record data field;
remotely obtain, by the study monitor client computing device, a digital copy of a clinical trial source data record,
the clinical trial source data record comprising, in a non-machine-readable format, a subject identifier corresponding to said case record identifier, a first source data field allegedly corresponding to said first case record data field, and a first source data value allegedly corresponding to said first case record data value;
convert at least a portion of the digital copy of the clinical trial source data record into a machine-readable format, such that the first source data field and the first source data value are computer-recognized;
compare the computer-recognized version of said first source data field with said first case record data field;
determine that the computer-recognized version of said first source data field corresponds to said first case record data field;
compare the computer-recognized version of said first source data value with said first case record data value;
determine that the computer-recognized version of said first source data value does not correspond to said first case record data value;
present to a study monitor, via the study monitor client computing device, digital case report form data including at least one of said first source data value and said first case record data value; and
generate, on the study monitor client computing device, a remote visual mismatch indicator affiliated with at least one of said first source data field, said first source data value, said first case record data field, and said first case record data value,
such that the remote visual mismatch indicator distinguishes, among digital case report form record data values, a record data value that the computing system automatically identified as not corresponding to the clinical trial source data record.

14. The non-transient computer-readable storage medium of claim 13, wherein the instructions to convert at least a portion of the digital copy of the clinical trial source data record into a machine-readable format are instructions to perform recognition selected from at least one of optical character recognition and voice recognition.

15. The non-transient computer-readable storage medium of claim 13, wherein the computer-recognized version of the first source data field or value comprises transcription confidence data, and wherein said presenting digital case report form data to a study monitor further comprises, if the transcription confidence is below a pre-defined threshold value, presenting the transcription confidence data or flagging the digital case report form data for manual validation.

16. The non-transient computer-readable storage medium of claim 13, where said digital copy of a clinical trial source data record comprises a certified digital copy of the clinical trial source data record.

17. The non-transient computer-readable storage medium of claim 13, containing further instructions to:
send, from the study monitor client computing device, an update request for said first case record data value;
obtain, by the study monitor client computing device, an updated digital case report form having an updated first case record data value;
compare the computer-recognized version of said first source data value with said updated first case record data value; and
determine that the computer-recognized version of said first source data value does correspond to said updated first case record data value.

18. The non-transient computer-readable storage medium of claim 13, containing further instructions to add a metatag to at least one of the digital case report form and said clinical trial source data record.

19. A clinical trial facilitated remote verification method performed by a computing system, the method comprising:
obtaining, by a study monitor client computing device, a digital case report form,
the digital case report form comprising a case record identifier, a first case record data field, and a first case record data value associated with the first case record data field;
obtaining, by the study monitor client computing device, a certified digital copy of a clinical trial source data record,
the clinical trial source data record comprising, in a non-machine-readable format, a subject identifier corresponding to said case record identifier, a first source data field allegedly corresponding to said first case record data field, and a first source data value allegedly corresponding to said first case record data value;
converting at least a portion of the certified digital copy of the clinical trial source data record into a machine-readable format, such that the first source data field and the first source data value are computer-recognized;
comparing the computer-recognized version of said first source data field with said first case record data field;
determining that the computer-recognized version of said first source data field corresponds to said first case record data field;
comparing the computer-recognized version of said first source data value with said first case record data value;
determining that the computer-recognized version of said first source data value corresponds to said first case record data value;
determining that the computer-recognized version of said first source data value is outside a predetermined first source data value range;
presenting to a study monitor, via the study monitor client computing device, digital case report form data including at least one of said first source data value and said first case record data value; and
generating, on the study monitor client computing device, a visual out-of-bounds indicator affiliated with at least one of said first source data field, said first source data value, said first case record data field, and said first case record data value,
such that the visual out-of-bounds indicator distinguishes, among digital case report form record data values, a record data value that the computing system automatically identified as outside of a predetermined reference range.

20. The method of claim 19, further comprising:
sending, from the study monitor client computing device, an update request for said first source data value;
obtaining, by the study monitor client computing device, an updated certified digital copy of the clinical trial source data record having said updated first source data value;
converting at least a portion of the updated certified digital copy of the clinical trial source data record into a machine-readable format, such that said updated first source data value is computer-recognized;
determining that at least one of the computer-recognized version of said updated first source data value and said first case record data value is outside a predetermined updated first case record data value range; and
adding a metatag to at least one of the digital case report form and said clinical trial source data record.
